(19) United States
(12) Patent Application Publication
(10) Pub. No.: US 2019/0099062 A1
(43) Pub. Date: Apr. 4, 2019

(54) MEDICAL DEVICE

(71) Applicant: OLYMPUS CORPORATION, Tokyo (JP)

(72) Inventors: Yasushige ISHIHARA, Tokyo (JP); Kazutoshi KUMAGAI, Tokyo (JP); Naoya SUGIMOTO, Tokyo (JP)

(73) Assignee: OLYMPUS CORPORATION, Tokyo (JP)

(21) Appl. No.: 16/207,265

(22) Filed: Dec. 3, 2018

Related U.S. Application Data
(63) Continuation of application No. PCT/JP2016/067509, filed on Jun. 13, 2016.

(51) Int. Cl. A61B 1/005; A61B 17/04; A61B 1/00

(52) U.S. Cl. CPC A61B 1/0057; A61B 1/00135; A61B 1/00045; A61B 17/0469

(57) ABSTRACT

A medical device includes: a sheath that is provided with a flexible section configured to have flexibility so as to be deformed between a first shape extending along the longitudinal axis and a second shape bent in a direction intersecting the longitudinal axis; a deformation assisting member attached to the sheath so as to be movable along a surface of the sheath; a position adjustment part connected to a proximal end section of the sheath, the position adjustment part being configured to adjust a relative position of the deformation assisting member with respect to the sheath; and a curved section provided at one of the flexible section and one longitudinal section of the deformation assisting member and having a bent shape. The deformation assisting member disposed at the flexible section imparts to the flexible section a driving force for causing deformation between the first shape and the second shape.

### CROSS-REFERENCE TO RELATED APPLICATIONS

This is a continuation of International Application PCT/JP2016/067509 which is hereby incorporated by reference herein in its entirety.

### TECHNICAL FIELD

The present invention relates to a medical device.

### BACKGROUND ART

In the related art, there is a generally used endoscope that has, at a distal end section, a bending section for changing the direction of the field of view. The bending section is provided with, for example, a plurality of bending pieces that are pivotably connected and a drive wire that is connected to the bending pieces and that makes the bending pieces pivot. Such a bending mechanism has a complicated structure and is not suitable for use in a small-diameter, low-cost endoscope.

Thus, a mechanism for bending a distal end section of an endoscope by means of a more simple structure has been proposed (for example, see PTLs 1 and 2). PTL 1 discloses a sheath having a distal end section that is bent into an arc shape, and a distal end section of an endoscope inserted into the sheath is bent according to the shape of the distal end section of the sheath. PTL 2 discloses a stylet the distal end section of which can be bent, and the distal end section of the stylet is bent in a channel of an endoscope, thereby bending the distal end section of the endoscope.

### CITATION LIST

### Patent Literature

{PTL 1} U.S. Unexamined Patent Application Publication No. 2014/364901
{PTL 2} Japanese Examined Patent Application, Publication No. Hei 6-67377

### SUMMARY OF INVENTION

The present invention provides a medical device including: a sheath that has a longitudinal axis and that is provided with a flexible section provided at least at a distal end section of the sheath, the flexible section being configured to have flexibility so as to be deformed between a first shape extending along the longitudinal axis and a second shape bent in a direction intersecting the longitudinal axis; a deformation assisting member having a longitudinal axis extending parallel to the longitudinal axis of the sheath, the deformation assisting member being configured to be attached to the sheath so as to be movable along a surface of the sheath; a position adjustment part connected to a proximal end section of the sheath, the position adjustment part being configured to adjust a relative position of the deformation assisting member with respect to the sheath; and a curved section provided at one of the flexible section and one longitudinal section of the deformation assisting member and having a bent shape in a natural state, wherein the position adjustment part adjusts the relative position of the deformation assisting member with respect to the sheath, thereby causing the deformation assisting member to impart to the flexible section a driving force for causing deformation between the first shape and the second shape, according to an adjustment amount adjusted by the position adjustment part.

### BRIEF DESCRIPTION OF DRAWINGS

FIG. 1 is a view showing the overall configuration of a medical device according to a first embodiment of the present invention.

FIG. 2A is a view showing the configuration of a distal end section of the medical device shown in FIG. 1 when a distal end section of a sheath is straight.

FIG. 2B is a view showing the configuration of the distal end section of the medical device shown in FIG. 1 when the distal end section of the sheath is bent.

FIG. 2C is a cross-sectional view cut along the II-II line of FIG. 2A.

FIG. 3 is a view for explaining how to use the medical device shown in FIG. 1.

FIG. 4A is a view showing the configuration of a distal end section of the medical device shown in FIG. 1 according to a first modification when the distal end section of the sheath is bent.

FIG. 4B is a view showing the configuration of the distal end section of the medical device shown in FIG. 1 according to the first modification when the distal end section of the sheath is straight.

FIG. 5A is a view showing the configuration of a distal end section of the medical device shown in FIG. 1 according to a second modification when the distal end section of the sheath is straight.

FIG. 5B is a view showing the configuration of the distal end section of the medical device shown in FIG. 1 according to the second modification when the distal end section of the sheath is bent.

FIG. 6 is a view showing a distal end section of the medical device shown in FIG. 1 according to a third modification.

FIG. 7A is a view showing the configuration of a distal end section of the medical device shown in FIG. 1 according to a fourth modification when the distal end section of the sheath is straight.

FIG. 7B is a view showing the configuration of the distal end section of the medical device shown in FIG. 1 according to the fourth modification when the distal end section of the sheath is bent.

FIG. 8A is a view showing the configuration of a distal end section of the medical device shown in FIG. 7A according to a modification when the distal end section of the sheath is straight.

FIG. 8B is a view showing the configuration of the distal end section of the medical device shown in FIG. 7A according to the modification when the distal end section of the sheath is bent.

FIG. 9 is a view showing the configuration of a distal end section of the medical device shown in FIG. 1 according to a fifth modification.

FIG. 10 is a view showing the configuration of a distal end section of the medical device shown in FIG. 1 according to a sixth modification.

FIG. 11 is a view showing the configuration of a distal end section of a medical device according to a second embodiment of the present invention.

FIG. 12 is a view showing the configuration of a treatment tool provided in the medical device shown in FIG. 11.

FIG. 13A is a view showing the configuration of a distal end section of the medical device shown in FIG. 11 according to a modification when the distal end section of the sheath is bent.

FIG. 13B is a view showing the configuration of the distal end section of the medical device shown in FIG. 11 according to the modification when the distal end section of the sheath is straight.

FIG. 14A is a view showing the overall configuration of the medical device shown in FIGS. 13A and 13B.

FIG. 14B is a view showing the overall configuration of the medical device shown in FIGS. 13A and 13B.

FIG. 15 is a view showing the configuration of a distal end section of a medical device according to a third embodiment of the present invention.

FIG. 16 is a view showing the configuration of a guide wire provided in the medical device shown in FIG. 15.

FIG. 17A is a view showing the configuration of a distal end section of a medical device according to a fourth embodiment of the present invention when the distal end section of the sheath is straight.

FIG. 17B is a view showing the configuration of the distal end section of the medical device according to the fourth embodiment of the present invention when the distal end section of the sheath is bent.

FIG. 18 is a view showing the configuration of the medical device shown in FIGS. 17A and 17B according to a modification.

FIG. 19 is a view showing a distal end section of the medical device shown in FIG. 1 according to another modification.

### DESCRIPTION OF EMBODIMENTS

### First Embodiment

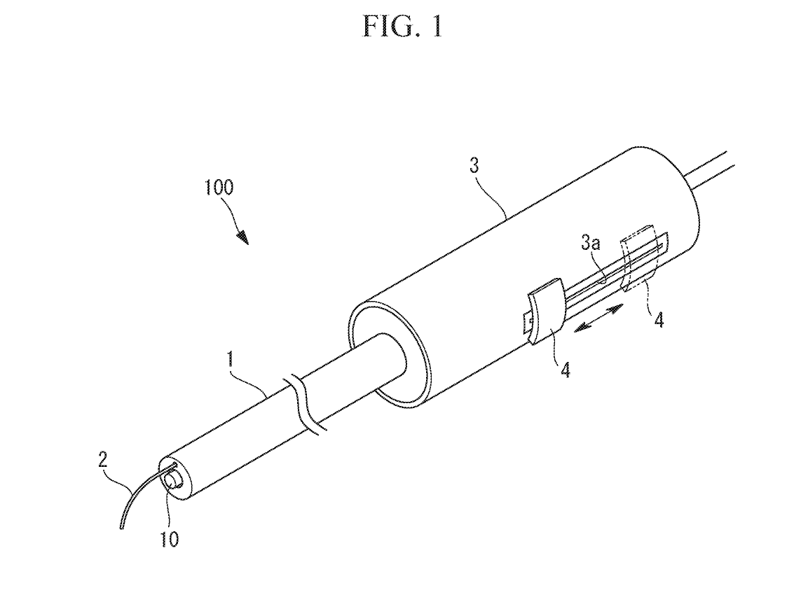

A medical device 100 according to a first embodiment of the present invention will be described with reference to FIGS. 1 to 10.

The medical device 100 of this embodiment is used when attached to an endoscope 10 and, as shown in FIG. 1, is provided with: a sheath (insertion portion) 1; a guide wire (deformation assisting member) 2 that is inserted into the sheath 1; and a gripped part 3 that is connected to the proximal end of the sheath 1.

The endoscope 10 is a flexible endoscope that has higher flexibility than the sheath 1 and that can be bent along the shape of the sheath 1. The endoscope 10 does not have a bending section for deflecting a distal end surface of the endoscope 10 and has a simple configuration that includes, therein, an illumination optical system for illuminating the field of view and an image acquisition optical system for receiving light from a subject and acquiring an image of the subject.

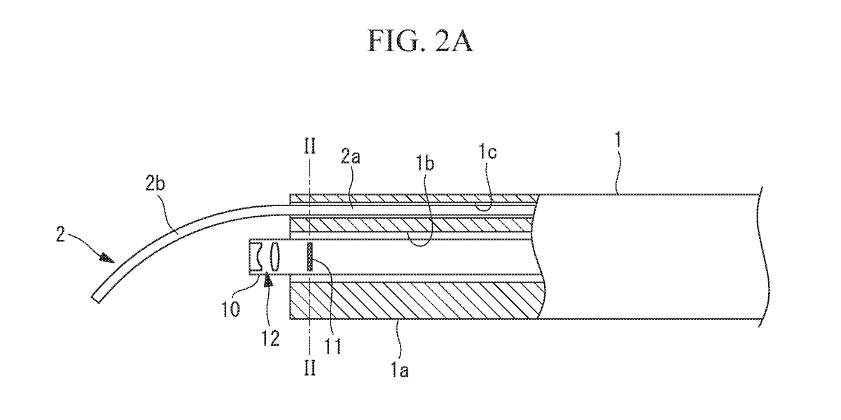

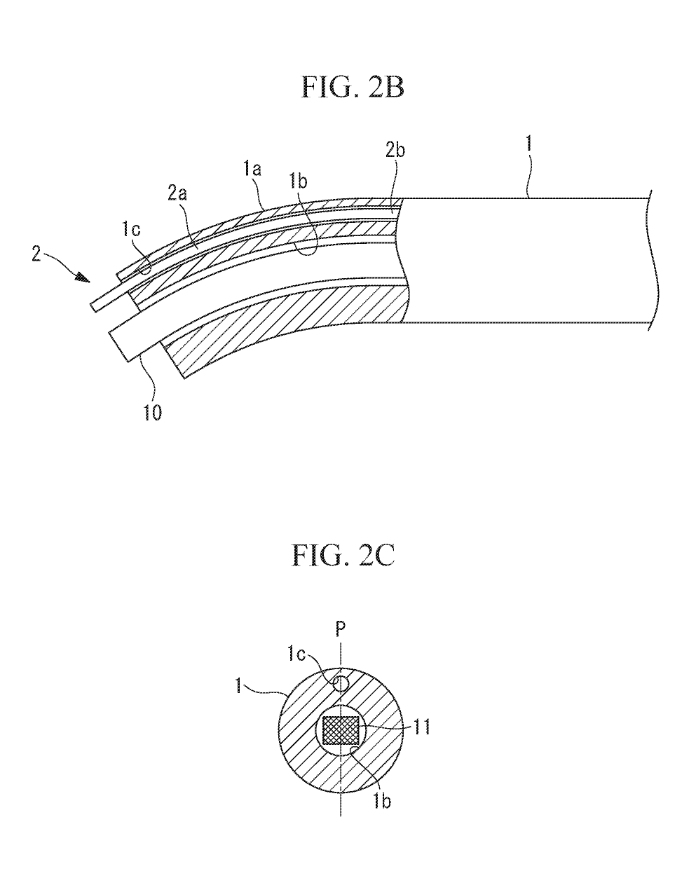

The sheath 1 is a cylindrical member having a longitudinal axis and has a straight shape (first shape) extending along the longitudinal axis over the entire length, in a natural state in which an external force does not act thereon. The sheath 1 has, at least at a distal end section (flexible section) 1a, flexibility so as to be deformable from the straight shape (see FIG. 2A), which extends along the longitudinal axis, to an arc shape (second shape, see FIG. 2B) that is bent in a direction intersecting the longitudinal axis. As shown in FIGS. 2A to 2C, the sheath 1 has an endoscope insertion path 1b and a wire insertion path (deformation-assisting-member insertion path) 1c that penetrate therethrough in the longitudinal direction. In FIG. 2A, reference sign 11 denotes an image acquisition device, such as a CCD or a CMOS image sensor, provided at a distal end section of the endoscope 10, and reference sign 12 denotes an objective lens that forms an image on the image acquisition device 11.

The guide wire 2 has a smaller outer diameter than the inner diameter of the wire insertion path 1c. The guide wire 2 is inserted into the wire insertion path 1c in the longitudinal direction, thereby being attached to the inside of the sheath 1 such that the longitudinal axis of the guide wire 2 is parallel to the longitudinal axis of the sheath 1. The guide wire 2 disposed inside the wire insertion path 1c is movable in the longitudinal direction of the sheath 1 along an inner surface of the wire insertion path 1c.

The guide wire 2 has a straight section 2a that has a longitudinal axis and a bending section (curved section) 2b that is connected to a distal end of the straight section 2a. The straight section 2a has a straight shape extending along the longitudinal axis. The bending section 2b is bent in a direction intersecting the longitudinal axis of the straight section 2a. The bending section 2b is disposed in the distal end section 1a of the sheath 1 or in the vicinity of the distal end section 1a.

The guide wire 2 has, at least at the bending section 2b, larger rigidity than the distal end section 1a of the sheath 1 in a state in which the distal end section of the endoscope 10 inserted into the endoscope insertion path 1b is disposed in the distal end section 1a of the sheath 1. Therefore, as shown in FIG. 2A, when the entire bending section 2b is protruded from the distal end of the sheath 1 and is disposed outside the sheath 1, the distal end section 1a of the sheath 1 and the distal end section of the endoscope 10 are formed into a straight shape. On the other hand, as shown in FIG. 2B, when the entire bending section 2b is disposed in the distal end section 1a of the sheath 1, the bending section 2b, which has larger rigidity, imparts to the distal end section 1a of the sheath 1 a driving force for bending the distal end section 1a, thus forming the distal end section 1a of the sheath 1 and the distal end section of the endoscope 10 into an arc shape according to the shape of the bending section 2b.

The gripped part 3 is a part gripped by an operator when the operator operates the medical device 100. The gripped part 3 is provided with a position adjustment part 4 that adjusts the position of the bending section 2b in the longitudinal direction with respect to the distal end section 1a of the sheath 1 by moving the guide wire 2 in the wire insertion path 1c. The proximal end section of the guide wire 2 extends from the proximal end of the wire insertion path 1c to the gripped part 3 and is connected to the position adjustment part 4. A slot 3a that extends in the longitudinal direction of the sheath 1 is formed in the gripped part 3. The position adjustment part 4 is provided so as to be movable along the slot 3a. Therefore, the guide wire 2 is moved toward the distal end with respect to the sheath 1 when the position adjustment part 4 is moved toward the distal end, and the guide wire 2 is moved toward the proximal end with respect to the sheath 1 when the position adjustment part 4 is moved toward the proximal end.

Here, the position adjustment part 4 is provided so as to be continuously movable along the slot 3a between a first position (see the solid line in FIG. 1) at which the entire bending section 2b is protruded from the distal end of the sheath 1, thus being disposed outside the sheath 1, and a second position (see the two-dot chain line in FIG. 1) at which the entire bending section 2b is disposed in the distal end section 1a of the sheath 1, thus being accommodated in the wire insertion path 1c. Accordingly, the operator can continuously change the bending angle of the distal end section 1a of the sheath 1 between the straight shape and the arc shape by using the position adjustment part 4, which is located close at hand.

Next, the operation of the thus-configured medical device 100 will now be described.

In this embodiment, although a description will be given of a case in which the inside of a pericardial cavity is observed, the intended use of the medical device 100 is not limited to observation of the pericardial cavity, and the medical device 100 can be used to observe another site in the body.

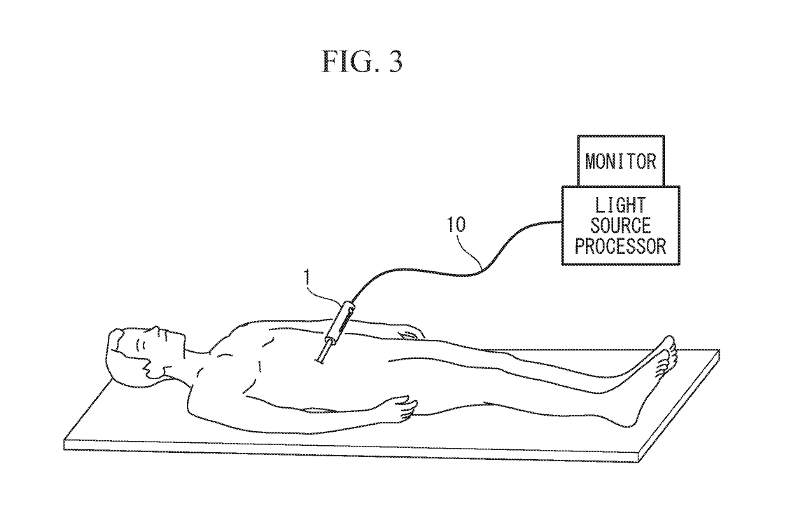

The sheath 1, in which the guide wire 2 is attached to the inside of the wire insertion path 1c, is inserted into the body from under the xiphoid process, as shown in FIG. 3, and is disposed in the pericardial cavity. Next, the endoscope 10 is inserted into the endoscope insertion path 1b, and the distal end section of the endoscope 10 is disposed in the distal end section of the endoscope insertion path 1b. Accordingly, the interior of the pericardial cavity can be observed by means of the endoscope 10.

At this time, the operator operates the position adjustment part 4, which is located close at hand, between the second position and the first position, thereby making it possible to control bending of the distal end section 1a of the sheath 1. Specifically, the operator operates the position adjustment part 4 to the first position, thereby forming the distal end section 1a of the sheath 1 and the distal end section of the endoscope into a straight shape, and making it possible to observe a forward field of view in the longitudinal direction of the sheath 1 by means of the endoscope 10. Furthermore, the operator operates the position adjustment part 4 to the second position, thereby bending the distal end section 1a of the sheath 1 and the distal end section of the endoscope 10 into an arc shape, and making it possible to observe an oblique direction with respect to the longitudinal direction of the sheath 1 by means of the endoscope 10. Furthermore, the operator adjusts the position of the position adjustment part 4 between the first position and the second position, thereby making it possible to adjust the bending angle of the distal end section 1a of the sheath 1.

In this way, according to this embodiment, by means of a simple configuration in which the bending section 2b, which has larger rigidity than the distal end section 1a of the sheath 1, is provided at the distal end section of the guide wire 2, and the bending section 2b is merely moved in the longitudinal direction with respect to the distal end section 1a of the sheath 1, it is possible to control bending of the distal end section of the endoscope 10 disposed in the distal end section 1a of the sheath 1. Accordingly, there is an advantage in that it is possible to realize a smaller-diameter, lower-cost medical device 100, compared with an endoscope that is provided with a complicated bending mechanism. Furthermore, in a state in which the bending section 2b is protruded from the distal end of the sheath 1, shown in FIG. 2A, the pericardium is held at a position away from the distal end surface of the sheath 1 in the forward direction, due to the bending section 2b, and a space is formed in the vicinity of the distal end of the sheath 1 where the distal end of the endoscope 10 is disposed. Accordingly, there is an advantage in that the field of view of the endoscope 10 can be ensured in the pericardial cavity.

In this embodiment, it is preferable that a means for determining a rotational position of the guide wire 2 about the longitudinal axis in the wire insertion path 1c be provided such that the bending section 2b of the guide wire 2 is bent in a plane P (see FIG. 2C) that passes through the central axis of the endoscope insertion path 1b and the central axis of the wire insertion path 1c. Furthermore, it is preferable that a fixing means for fixing the endoscope 10 about the longitudinal axis with respect to the sheath 1 be provided such that the up-and-down direction of an endoscopic image acquired by the endoscope 10 matches the bending direction of the distal end section 1a of the sheath 1 caused by the bending section 2b. For example, it is preferable that the central axis of the wire insertion path 1c be positioned on the center line of the image acquisition device 11 in the right-and-left direction (right and left in the plane of the figure). At this time, the bending section 2b of the guide wire 2 is bent in the up-and-down direction of the image acquisition device 11.

By doing so, the field of view of the endoscope 10 can be changed in the up-and-down direction of the endoscopic image through bending of the distal end section 1a of the sheath 1, thus making it possible to improve the usability.

The medical device 100 of this embodiment may be modified as follows.

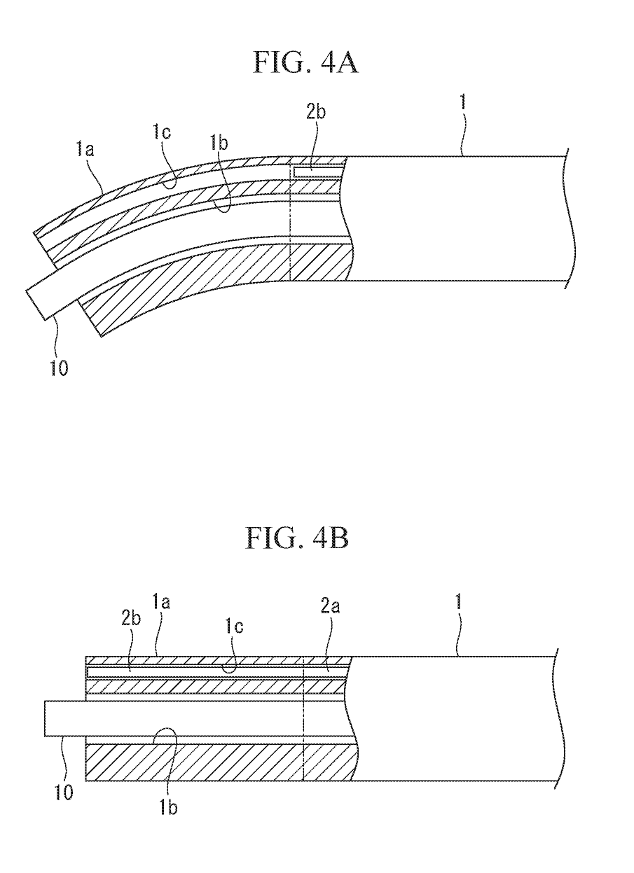

In a first modification of this embodiment, as shown in FIGS. 4A and 4B, the bending section is formed of the distal end section (curved section) 1a of the sheath 1, instead of the distal end section of the guide wire 2. The distal end section 1a of the sheath 1 has an arc shape in the natural state and is deformable into a straight shape due to the flexibility thereof. The guide wire 2 has a straight shape extending along the longitudinal axis over the entire length.

In this modification, as shown in FIG. 4A, when the distal end of the guide wire 2 is disposed closer to the proximal end than the distal end section 1a of the sheath 1 is, the distal end section 1a of the sheath 1 is formed into the arc shape. On the other hand, as shown in FIG. 4B, when the guide wire 2 is disposed in the distal end section 1a of the sheath 1, the distal end section 1a of the sheath 1 is formed into the straight shape.

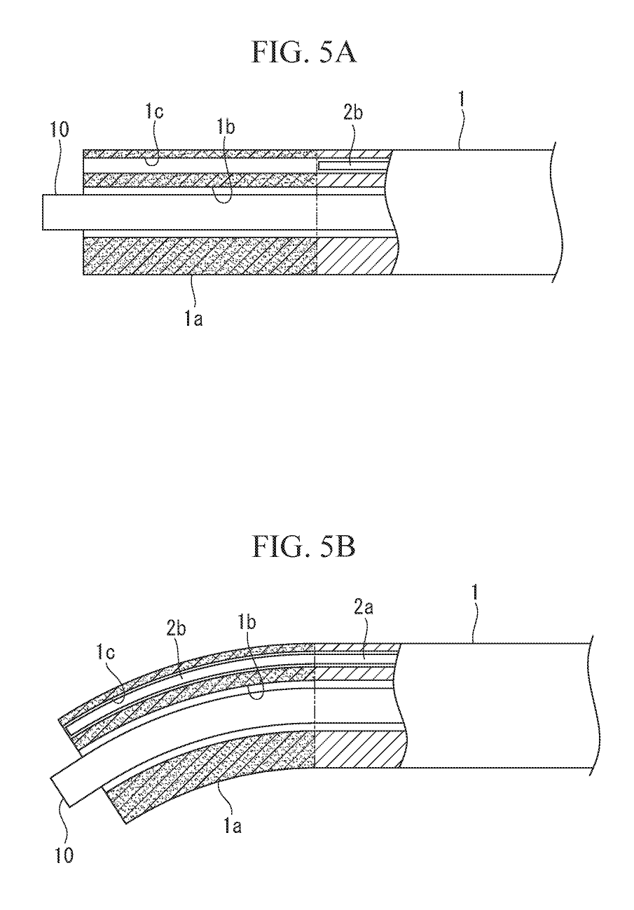

In a second modification of this embodiment, as shown in FIGS. 5A and 5B, the sheath 1 has, at the distal end section 1a, lower rigidity than the guide wire 2 and has, at a section closer to the proximal end than the distal end section 1a is, higher rigidity than the guide wire 2. The guide wire 2 has a straight section 2a that has a longitudinal axis and a bending section (curved section) 2b that is connected to a distal end of the straight section 2a. The straight section 2a has a straight shape extending along the longitudinal axis. The bending section 2b is bent in a direction intersecting the longitudinal axis of the straight section 2a.

In this modification, as shown in FIG. 5A, when the distal end of the guide wire 2 is disposed closer to the proximal end than the distal end section 1a of the sheath 1 is, the distal end section 1a of the sheath 1 is formed into a straight shape. On the other hand, as shown in FIG. 5B, when the bending section 2b of the guide wire 2 is disposed in the distal end section 1a of the sheath 1, the distal end section 1a of the sheath 1 is formed into an arc shape.

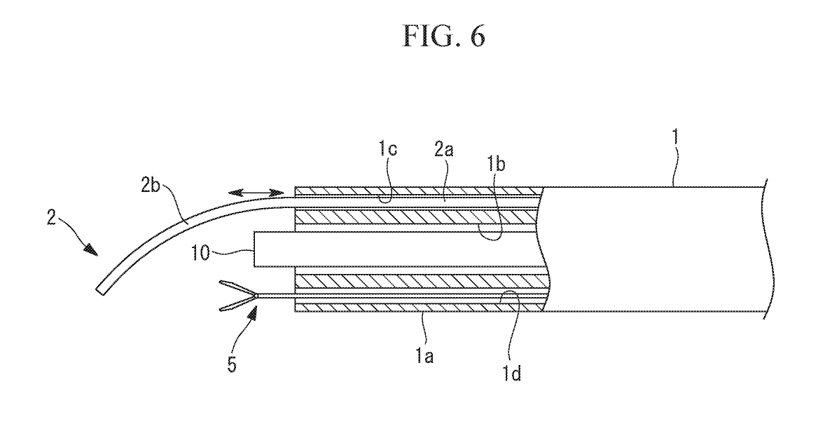

In a third modification of this embodiment, as shown in FIG. 6, the sheath 1 further has a treatment-tool insertion path 1d that penetrates therethrough in the longitudinal direction and into which a flexible treatment tool (for example, forceps) 5 is inserted in the longitudinal direction. In this modification, the bending section (curved section) 2b of the guide wire 2 has larger rigidity than the rigidity of the distal end section 1a in a state in which the endoscope 10 and the treatment tool 5 are disposed in the distal end section 1a of the sheath 1.

According to this modification, even if the distal end section 1a of the sheath 1 is deformed between the straight shape and the arc shape, because the relative positions of the distal end of the endoscope 10 and the distal end of the treatment tool 5 are maintained, the distal end of the treatment tool 5 can be observed always from the same direction by means of the endoscope 10. Furthermore, although the outer diameter of the sheath 1 is increased through addition of the treatment-tool insertion path 1d, it is possible to realize a smaller-diameter medical device, compared with a case in which a treatment-tool channel is provided in an endoscope that is provided with a complicated bending mechanism.

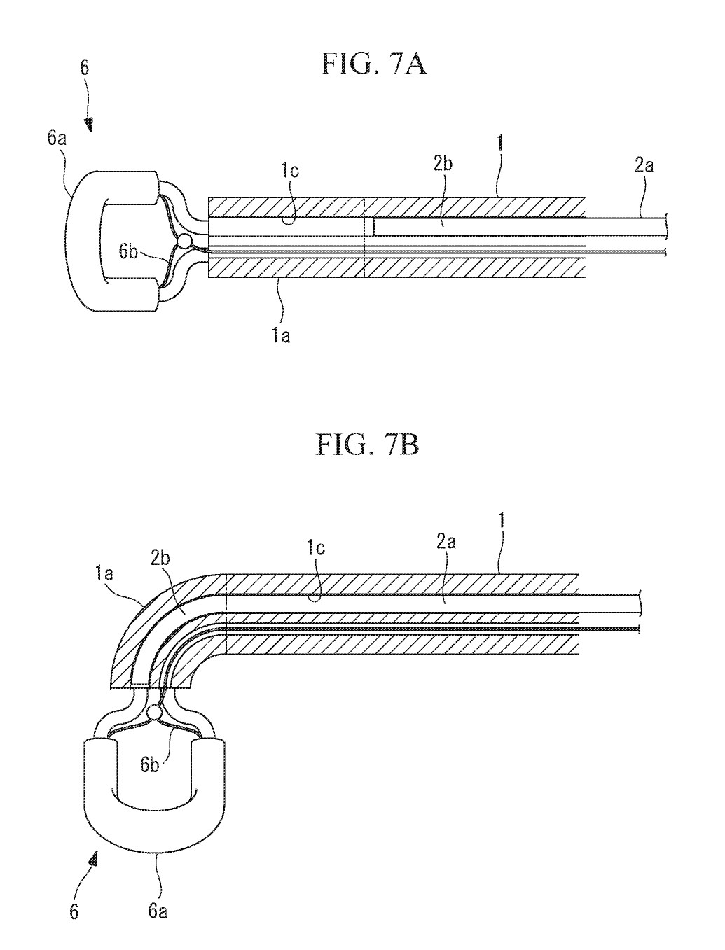

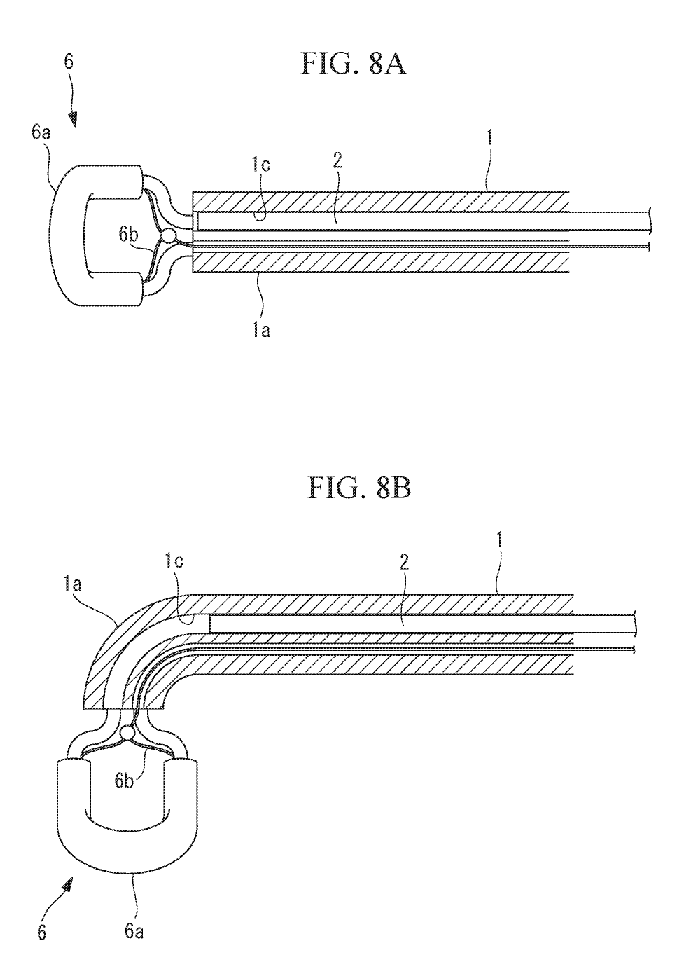

In a fourth modification of this embodiment, as shown in FIG. 7A to FIG. 8B, a left-atrial-appendage ligation device 6 that ligates the left atrial appendage of the heart is provided. The left-atrial-appendage ligation device 6 is provided with: a ring-like ligature extension part 6a that is fixed to the distal end of the sheath 1; and a ligature 6b that passes inside the ligature extension part 6a and that forms a loop at a position close to the distal end of the sheath 1. One end of the ligature 6b is disposed at the proximal end of the sheath 1 and is pulled, thereby reducing the diameter of the loop and making it possible to ligate the proximal of the left atrial appendage disposed inside the loop.

FIGS. 7A and 7B show an example case in which the left-atrial-appendage ligation device 6 is provided in a combination of the straight sheath 1 and the guide wire 2 that has the bending section 2b, as in the medical device shown in FIGS. 5A and 5B. FIGS. 8A and 8B show an example case in which the left-atrial-appendage ligation device 6 is provided in a combination of the sheath 1 that has the bent distal end section 1a and the guide wire 2 that has a straight shape extending along the longitudinal axis over the entire length, as in the medical device shown in FIGS. 4A and 4B.

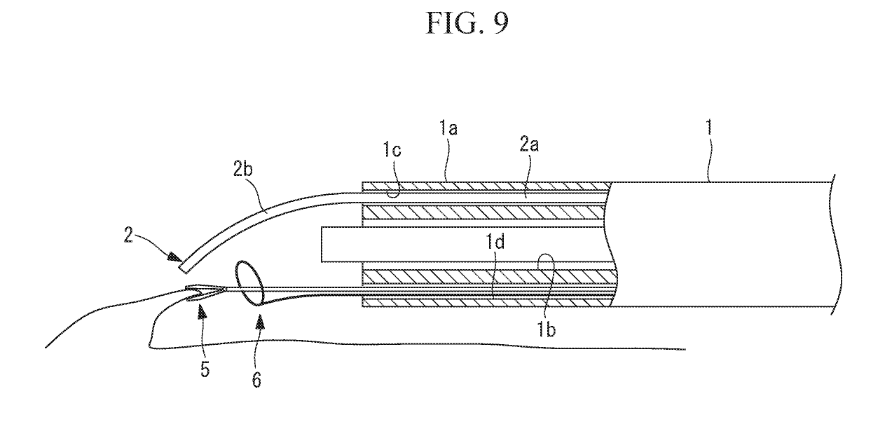

In a fifth modification of this embodiment, as shown in FIG. 9, the treatment tool 5 and the left-atrial-appendage ligation device 6 are both provided.

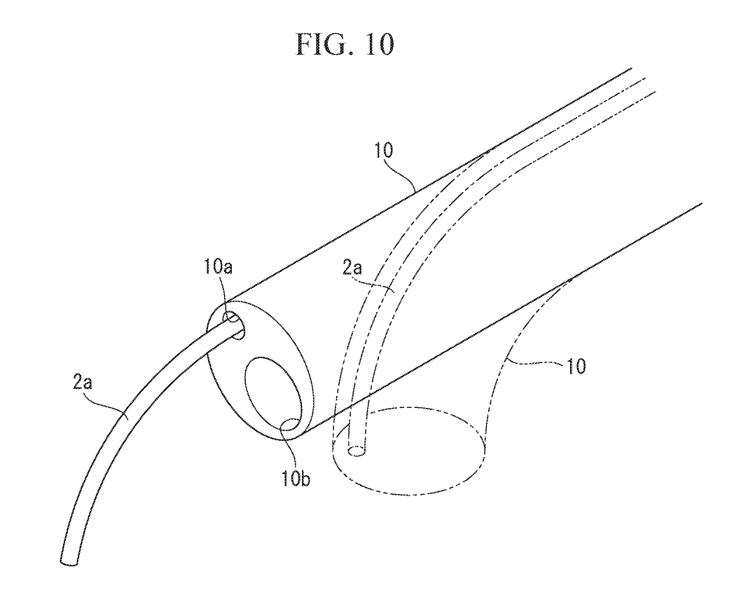

In a sixth modification of this embodiment, as shown in FIG. 10, an endoscope 10 that is provided with a wire channel 10a is used as the insertion portion, instead of the sheath 1. The endoscope 10 is provided with, as the image acquisition optical system, an objective lens 10b that is provided at the distal end of the endoscope 10 and that forms an image of light coming from a subject, and an image acquisition unit (not shown) that captures the image formed by the objective lens 10b, thus acquiring an endoscopic image.

The endoscope 10 has an up-and-down direction corresponding to the up-and-down direction of the endoscopic image. The wire channel 10a is provided at an upper side of the objective lens 10b, on the distal end surface of the endoscope 10. The guide wire 2 is provided in the wire channel 10a such that the bending direction of the bending section 2b matches the up-and-down direction of the endoscope 10.

By doing so, the distal end section of the endoscope 10 can be bent by the bending section 2b of the guide wire 2, in the up-and-down direction of an endoscopic image displayed on a monitor.

### Second Embodiment

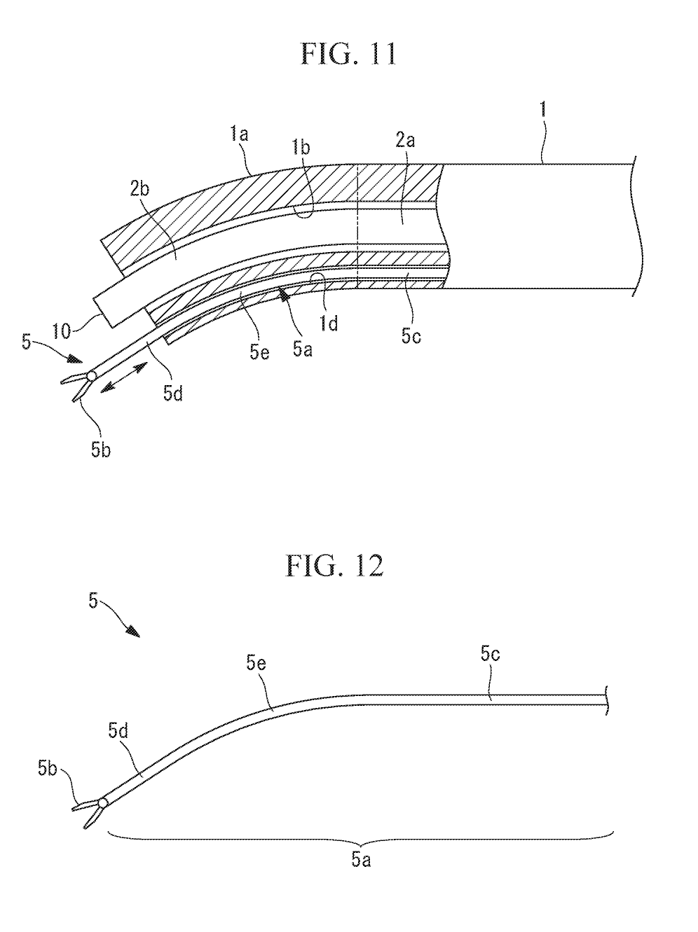

Next, a medical device according to a second embodiment of the present invention will be described with reference to FIG. 11 to FIG. 14B.

In this embodiment, a description will be mainly given of configurations that differ from those in the first embodiment; identical reference signs are assigned to configurations that are common to those in the first embodiment, and a description thereof will be omitted.

As shown in FIG. 11, the medical device of this embodiment mainly differs from that of the first embodiment in that a treatment tool (for example, grasping forceps) 5 is provided as the deformation assisting member, instead of the guide wire 2.

The sheath 1 has, instead of the wire insertion path 1c, a treatment-tool insertion path (deformation-assisting-member insertion path) 1d into which the treatment tool 5 is inserted.

As shown in FIG. 12, the treatment tool 5 has an elongated body section 5a that has a longitudinal axis and a distal-end operating part 5b that is provided at the distal end of the body section 5a and that is used to treat a living tissue. The body section 5a has a first straight section 5c that has a straight shape extending along the longitudinal axis, a second straight section 5d that is disposed closer to the distal end than the first straight section 5c is and that has a straight shape, and a bending section (curved section) 5e that connects the first straight section 5c and the second straight section 5d and that is bent in a direction intersecting the longitudinal axis.

The body section 5a has, at least at the bending section 5e, larger rigidity than the rigidity of the distal end section 1a in a state in which the distal end section of the endoscope 10 is disposed in the distal end section 1a of the sheath 1. Therefore, when the bending section 5e is protruded from the distal end of the sheath 1 and is disposed outside the sheath 1, the distal end section 1a of the sheath 1 and the distal end section of the endoscope 10 are formed into a straight shape. On the other hand, when the bending section 5e is disposed in the distal end section 1a of the sheath 1, the distal end section 1a of the sheath 1 and the distal end section of the endoscope 10 are bent into an arc shape according to the shape of the bending section 5e, due to the rigidity of the bending section 5e.

The proximal end section of the body section 5a extends from the proximal end of the treatment-tool insertion path 1d to the gripped part 3 and is connected to the position adjustment part 4. The position adjustment part 4 is provided so as to be continuously movable between the first position at which the entire bending section 5e is protruded from the distal end of the sheath 1, thus being disposed outside the sheath 1, and the second position at which the entire bending section 5e is disposed in the distal end section 1a of the sheath 1, thus being accommodated in the treatment-tool insertion path 1d. Therefore, as in the first embodiment, the operator can continuously change the bending angle of the distal end section 1a of the sheath 1 and the distal end section of the endoscope 10 between the straight shape and the arc shape, by using the position adjustment part 4, which is located close at hand.

According to this embodiment, the treatment tool 5 also serves as the deformation assisting member, thereby making it possible to achieve a reduction in the diameter and a reduction in the cost of the medical device. Furthermore, with the second straight section 5d, which is provided between the distal-end operating part 5b and the bending section 5e, the bending section 5e is disposed at a position away from the distal end of the treatment tool 5 toward the proximal end. Accordingly, even in a state in which the distal end section of the sheath 1 is bent into an arc shape, there is an advantage in that, with the distal-end operating part 5b, which is disposed at a position away from the distal end of the sheath 1 in the longitudinal direction, it is possible to ensure, in front of the distal end of the sheath 1 in the pericardial cavity, the field of view of the endoscope 10 and the space for treating the living tissue by means of the treatment tool 5.

Because the other effects of this embodiment are the same as those of the first embodiment, a description thereof will be omitted.

The medical device of this embodiment may be modified as follows.

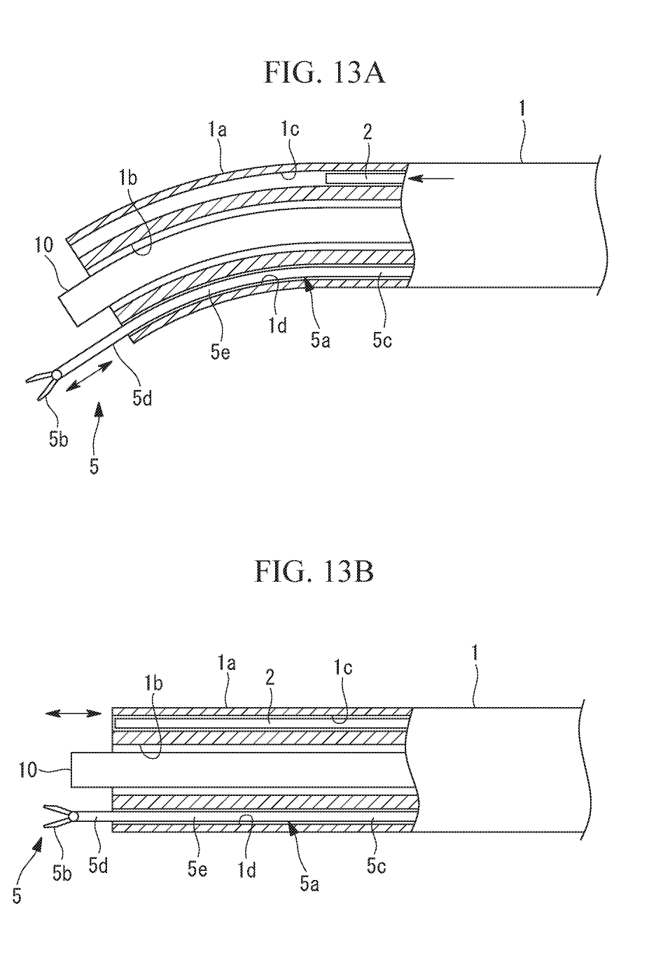

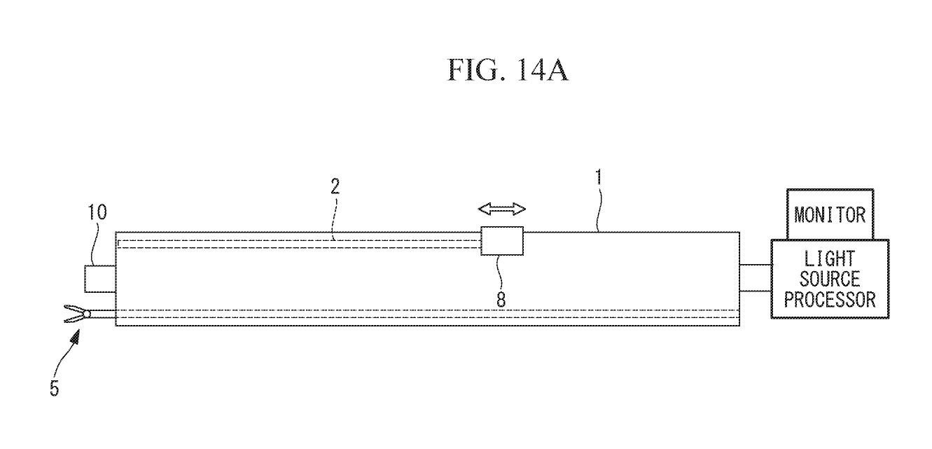

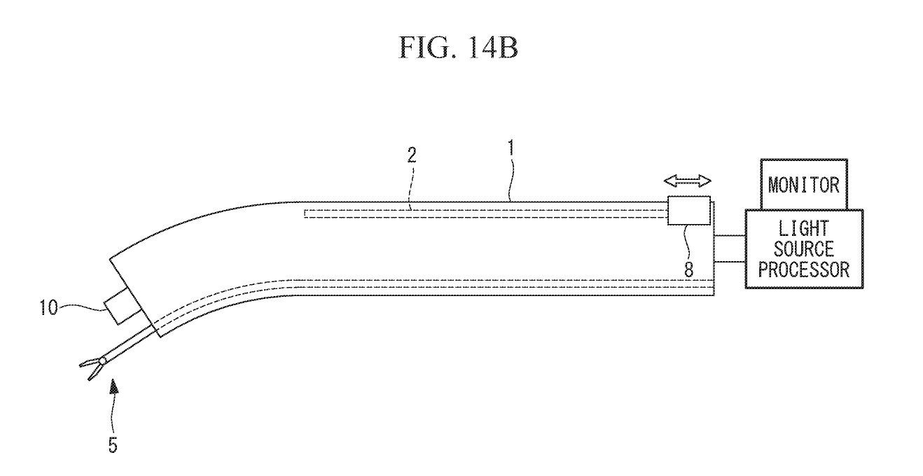

In a modification of this embodiment, as shown in FIGS. 13A and 13B, a guide wire 2 that has a straight shape over the entire length is further provided. The sheath 1 further has a wire insertion path 1c into which the guide wire 2 is inserted. The position of the guide wire 2 in the longitudinal direction is operated by means of an operation part 8 that is provided on the sheath 1, as shown in FIGS. 14A and 14B, for example. Although not shown in FIGS. 14A and 14B, an operation part for operating the distal-end operating part 5b is provided at the proximal end of the treatment tool 5.

In the medical device shown in FIG. 11, when the position of the bending section 5e with respect to the distal end section 1a is changed in order to fine-adjust the bending angle of the distal end section 1a of the sheath 1, the position of the distal-end operating part 5b is also changed. Therefore, for example, it is difficult to fine-adjust the bending angle of the distal end section 1a of the sheath 1 while grasping the living tissue by means of the distal-end operating part 5b.

In contrast to this, according to this modification, the position of the guide wire 2 with respect to the distal end section 1a of the sheath 1 is adjusted, thereby making it possible to fine-adjust the bending angle of the distal end section 1a of the sheath 1 while maintaining the position of the treatment tool 5 in the longitudinal direction with respect to the sheath 1 constant.

### Third Embodiment

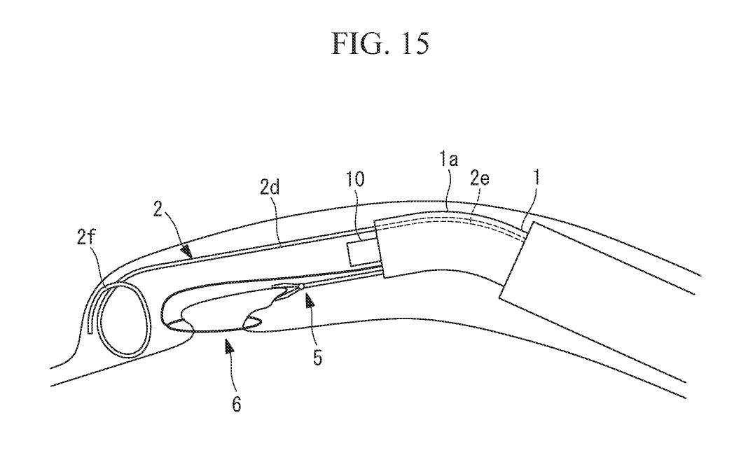

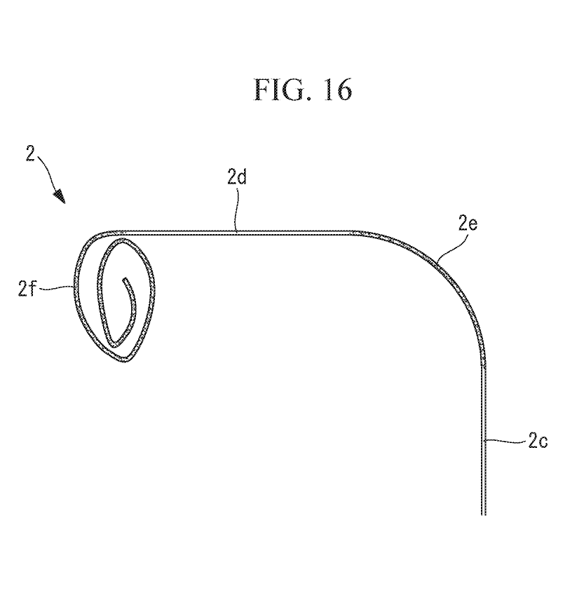

Next, a medical device according to a third embodiment of the present invention will be described with reference to FIGS. 15 and 16.

In this embodiment, a description will be mainly given of configurations that differ from those in the first embodiment; identical reference signs are assigned to configurations that are common to those in the first embodiment, and a description thereof will be omitted.

As shown in FIG. 15, the medical device of this embodiment differs from the medical device 100 of the first embodiment in terms of the shape of the guide wire 2.

As shown in FIG. 16, the guide wire 2 has: a first straight section 2c that has a straight shape extending along the longitudinal axis; a second straight section 2d that is located closer to the distal end than the first straight section 2c is and that has a straight shape; a bending section 2e that connects the first straight section 2c and the second straight section 2d and that is bent in a direction intersecting the longitudinal axis; and a loop section 2f that is connected to the distal end of the second straight section 2d and that has a loop shape. The loop section 2f is disposed in a direction intersecting the longitudinal direction of the second straight section 2d.

The position adjustment part 4 is provided so as to be continuously movable between a first position at which the entire bending section 2e is protruded from the distal end of the sheath 1, thus being disposed outside the sheath 1, and a second position at which the entire bending section 2e is disposed in the distal end section 1a of the sheath 1. Therefore, as in the first embodiment, the operator can continuously change the bending angle of the distal end section 1a of the sheath 1 and the distal end section of the endoscope 10 between the straight shape and the arc shape, by using the position adjustment part 4, which is located close at hand.

According to this embodiment, with the second straight section 2d, which is provided between the loop section 2f and the bending section 2e, the bending section 2e is disposed at a position away from the loop section 2f, which is provided at the distal end of the guide wire 2, toward the proximal end. Accordingly, even in a state in which the distal end section 1a of the sheath 1 is bent into an arc shape, there is an advantage in that, with the loop section 2f, which is disposed at a position away from the distal end of the sheath 1 in the longitudinal direction, and the second straight section 2d, which is provided between the bending section 2e and the loop section 2f, it is possible to hold the pericardium at a position away from the distal end surface of the sheath 1 in the forward direction and to ensure, in front of the distal end of the sheath 1 in the pericardial cavity, the field of view of the endoscope 10 and the space for treating the living tissue by means of the treatment tool 5 and the left-atrial-appendage ligation device 6. Furthermore, the loop section 2f is provided, thereby making it possible to ensure a larger space compared with a case in which the loop section 2f is not provided at the distal end of the guide wire 2. Because the other effects of this embodiment are the same as those of the first embodiment, a description thereof will be omitted.

### Fourth Embodiment

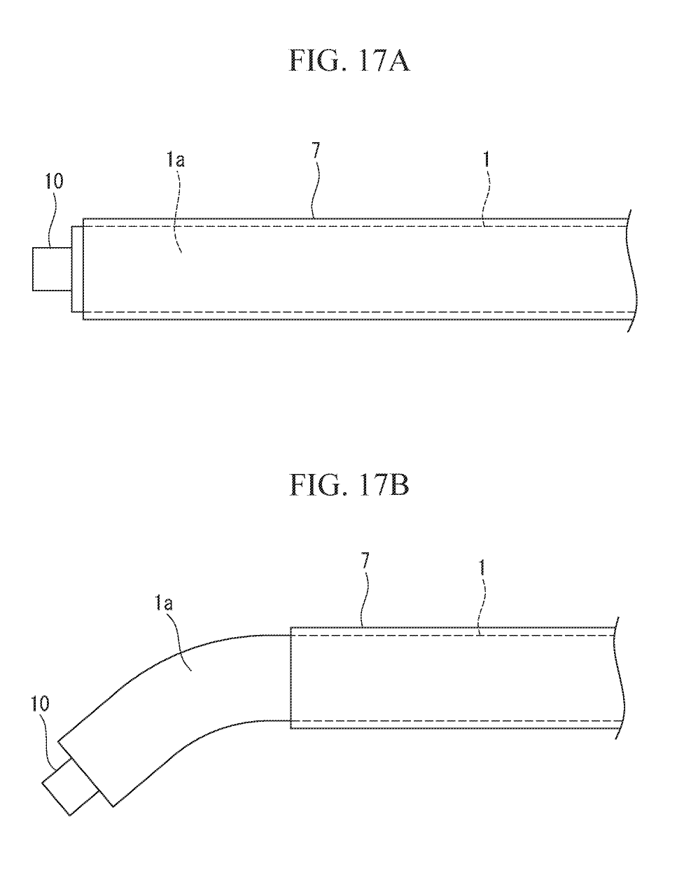

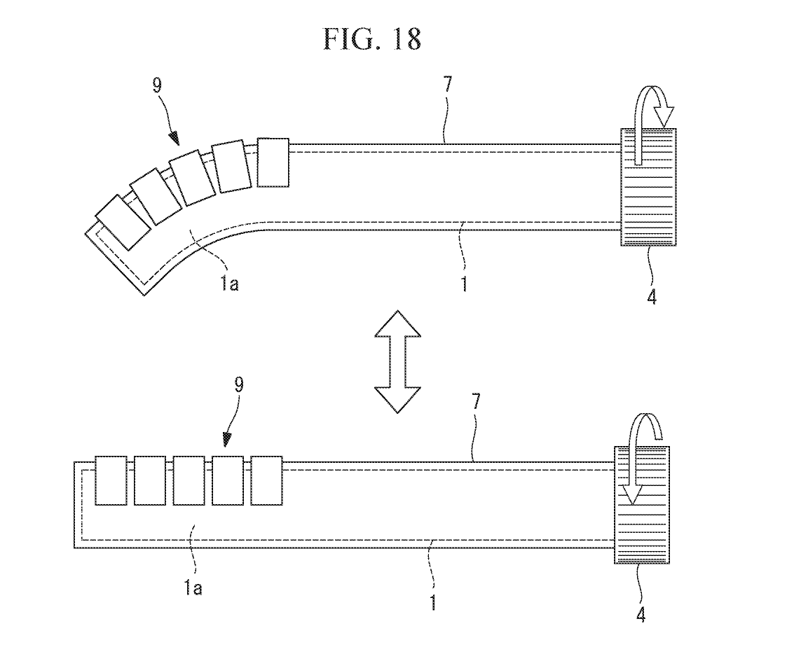

Next, a medical device according to a fourth embodiment of the present invention will be described with reference to FIG. 17A to FIG. 18.

In this embodiment, a description will be mainly given of configurations that differ from those in the first embodiment; identical reference signs are assigned to configurations that are common to those in the first embodiment, and a description thereof will be omitted.

As shown in FIGS. 17A and 17B, the medical device of this embodiment differs from the medical device 100 of the first embodiment in that a cylindrical outer sheath 7 that is disposed at an outer side of the sheath 1 is provided as the deformation assisting member, instead of the guide wire 2.

As in the first modification of the first embodiment, the bending section is formed of the distal end section (curved section) 1a of the sheath 1.

The outer sheath 7 has a straight shape extending along the longitudinal axis over the entire length, in the natural state. Furthermore, the outer sheath 7 has a larger inner diameter than the outer diameter of the sheath 1 and accommodates the sheath 1 in the direction along the longitudinal axis. The outer sheath 7 is movable with respect to the sheath 1 along the outer periphery of the sheath 1 in the longitudinal direction.

The outer sheath 7 has, at least at the distal end section, larger rigidity than the rigidity of the distal end section 1a in a state in which the distal end section of the endoscope 10 is disposed in the distal end section 1a of the sheath 1. Therefore, as shown in FIG. 17B, when the distal end of the outer sheath 7 is disposed closer to the proximal end than the distal end section 1a of the sheath 1 is, and the entire distal end section 1a of the sheath 1 is disposed outside the outer sheath 7, the distal end section 1a of the sheath 1 is formed into an arc shape. On the other hand, as shown in FIG. 17A, when the distal end of the outer sheath 7 is disposed at the same position as or in the vicinity of the distal end of the sheath 1, and almost the entire distal end section 1a of the sheath 1 is accommodated in the outer sheath 7, the distal end section 1a of the sheath 1 is formed into a straight shape due to the rigidity of the outer sheath 7.

The proximal end section of the outer sheath 7 is connected to the position adjustment part 4. The position adjustment part 4 is provided so as to be continuously movable between the first position and the second position, as in the first embodiment. In this embodiment, however, the first position is a position at which the distal end of the outer sheath 7 is disposed closer to the proximal end than the distal end section 1a of the sheath 1 is, and the second position is a position at which the distal end of the outer sheath 7 is disposed at the same position as or in the vicinity of the distal end of the sheath 1. Therefore, as in the first embodiment, the operator can continuously change the bending angle of the distal end section 1a of the sheath 1 and the distal end section of the endoscope 10 between the straight shape and the arc shape, by using the position adjustment part 4, which is located close at hand.

Because the other effects of this embodiment are the same as those of the first embodiment, a description thereof will be omitted.

The medical device of this embodiment may be modified as follows.

In a modification of this embodiment, as shown in FIG. 18, the outer sheath 7 has lower rigidity than the sheath 1, and the distal end section of the outer sheath 7 is configured so as to be capable of being bent only in one direction. The distal end section of the outer sheath 7 is disposed at the distal end section 1a of the sheath 1, and the position adjustment part 4 adjusts the rotational position of the outer sheath 7 about the longitudinal axis with respect to the sheath 1. In this configuration, when the direction in which the distal end section of the outer sheath 7 can be bent matches the bending direction of the distal end section 1a of the sheath 1 in the natural state, the distal end section 1a of the sheath 1 is bent into an arc shape.

A rigid member 9 that covers half the outer periphery of the distal end section of the outer sheath 7 and that is formed of a hard material is provided, for example, as a means for restricting the bending direction of the distal end section of the outer sheath 7 to only one direction. In the rigid member 9, notches extending in the circumferential direction of the outer sheath 7 are formed at intervals in the longitudinal direction. Due to the rigid member 9, the distal end section of the outer sheath 7 can be bent toward the opposite side from the side on which the rigid member 9 is provided but cannot be bent toward the same side as the side on which the rigid member 9 is provided. Instead of the rigid member 9, slits that extend in the circumferential direction may be formed in one circumferential section of the side wall of the outer sheath 7, at intervals in the direction along the longitudinal axis.

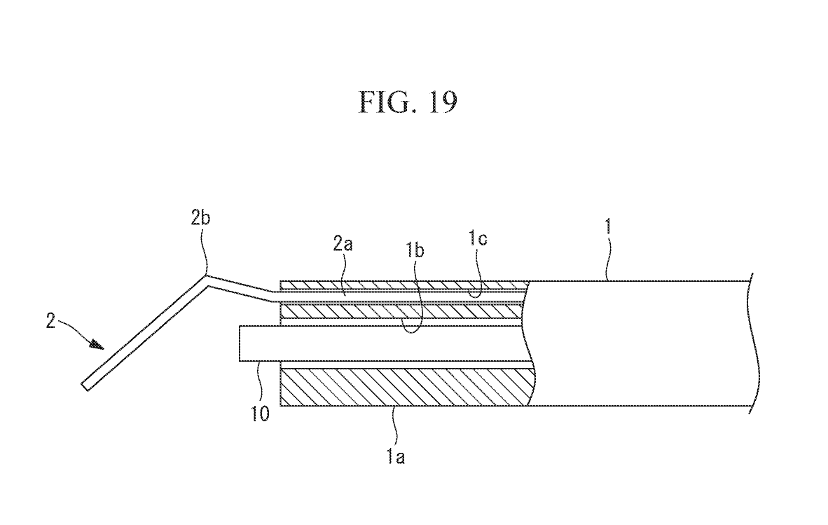

In the above-described first to fourth embodiments, although the sheath 1 is deformed between the straight-like first shape and the gently-bending arc-like second shape, the second shape is not limited to the arc shape and may be an inflected shape. In this case, the curved section 2b of the deformation assisting member 2 is also formed into an inflected shape, as shown in FIG. 19. Although FIG. 19 shows the configuration of the first embodiment, this modification can also be applied to the other embodiments.

The present invention is not limited to the above-described embodiments and modifications, and any combinations of the above-described embodiments and modifications are also encompassed in the present invention. For example, a combination of the left-atrial-appendage ligation device 6, which is shown in the fourth modification of the first embodiment, and the embodiment in which the medical device is used in a state in which the guide wire 2 is protruded from the distal end section of the sheath 1, as in the first embodiment, is also encompassed in the present invention. Furthermore, a combination of: the sheath 1 and the outer sheath 7, which are shown in the fourth embodiment; and the left-atrial-appendage ligation device 6, which is shown in the fourth modification of the first embodiment, or the treatment tool 5, which is shown in the third modification, is also encompassed in the present invention.

The above-described embodiment also leads to the following invention.

The present invention provides a medical device including: an insertion portion that has a longitudinal axis and that is provided with a flexible section provided at least at a distal end section of the insertion portion and having flexibility so as to be deformed between a first shape extending along the longitudinal axis and a second shape bent in a direction intersecting the longitudinal axis; a deformation assisting member that has a longitudinal axis extending parallel to the longitudinal axis of the insertion portion and that is attached to the insertion portion so as to be movable along a surface of the insertion portion; a position adjustment part that is connected to a proximal end section of the insertion portion and that adjusts a relative position of the deformation assisting member with respect to the insertion portion; and a curved section that is provided at one of the flexible section and one longitudinal section of the deformation assisting member and that has a bent shape in a natural state, wherein the position adjustment part adjusts the relative position of the deformation assisting member with respect to the insertion portion, thereby causing the deformation assisting member to impart to the flexible section a driving force for causing deformation between the first shape and the second shape, according to an adjustment amount adjusted by the position adjustment part.

According to the present invention, the position adjustment part adjusts the position of the deformation assisting member with respect to the flexible section, thereby making it possible to deform the flexible section between the first shape and the second shape. Specifically, in a case in which the curved section is provided at the flexible section, the deformation assisting member imparts to the flexible section a driving force for causing deformation from the second shape to the first shape. On the other hand, in a case in which the curved section is provided at one section of the deformation assisting member, the deformation assisting member imparts to the flexible section a driving force for causing deformation from the first shape to the second shape. In this way, by means of a simple structure in which the curved section is provided in one of the insertion portion and the deformation assisting member, and the relative positions of the insertion portion and the deformation assisting member are merely adjusted, it is possible to adjust the bending angle of the distal end section.

In the above-described invention, the deformation assisting member may have larger rigidity than the rigidity of the flexible section.

By doing so, the deformation assisting member is disposed in the flexible section, thereby making it possible to deform the flexible section according to the shape of the deformation assisting member.

In the above-described invention, the flexible section may have the first shape in the natural state; and the curved section may be provided at the one longitudinal section of the deformation assisting member.

By doing so, the curved section of the deformation assisting member is disposed in the flexible section, thereby making it possible to deform the distal end section of the insertion portion from a straight shape to a bent shape.

In the above-described invention, the flexible section may have the second shape in the natural state; the curved section may be provided at one section or the entire section of the flexible section; and the deformation assisting member may have a straight shape extending along the longitudinal axis of the deformation assisting member over the entire length.

By doing so, the deformation assisting member is disposed in the flexible section, thereby making it possible to deform the distal end section of the insertion portion from a bent shape to a straight shape.

In the above-described invention, the insertion portion may have an insertion path that penetrates therethrough in a longitudinal direction and into which the deformation assisting member is inserted; and the deformation assisting member may have the curved section at a position away from the distal end section toward the proximal end. In this case, the deformation assisting member may be a treatment tool that has, at a position closer to the distal end than the curved section is, a distal-end operating part that treats a living tissue or may be a wire having, at a position closer to the distal end than the curved section is, a loop section that is disposed in a direction intersecting the longitudinal direction of the insertion portion and that is formed into a loop shape.

By doing so, in a state in which the distal end section of the insertion portion is bent, it is possible to ensure a space in front of the distal end of the insertion portion even in a narrow body cavity, such as a pericardial cavity, due to a section of the deformation assisting member that is closer to the distal end than the curved section thereof is and that is disposed with being protruded from the distal end of the insertion portion in the longitudinal direction.

In the above-described invention, the insertion portion may have: a deformation-assisting-member insertion path that penetrates therethrough in a longitudinal direction and into which the deformation assisting member is inserted; and an endoscope insertion path that penetrates therethrough in the longitudinal direction and into which an endoscope is inserted; and the curved section may be provided so as to be bent in a plane that includes a central axis of the deformation-assisting-member insertion path and a central axis of the endoscope insertion path.

The endoscope has an up-and-down direction corresponding to the up-and-down direction of an endoscopic image acquired by the endoscope. The endoscope is disposed in the endoscope insertion path such that the deformation-assisting-member insertion path is positioned above or below the endoscope, thereby making it possible to bend the distal end section of the endoscope disposed in the distal end section of the insertion portion, in the up-and-down direction of the endoscopic image.

In the above-described invention, the insertion portion may have an endoscope insertion path that penetrates therethrough in a longitudinal direction and into which an endoscope is inserted; and a fixing means for fixing the endoscope in the endoscope insertion path about a longitudinal axis of the endoscope with respect to the insertion portion may be further provided.

By doing so, the fixing means can fix the endoscope in the endoscope insertion path such that the bending direction of the distal end section of the insertion portion becomes a predetermined direction with respect to the up-and-down and right-and-left directions of an endoscopic image.

### REFERENCE SIGNS LIST

1 sheath (insertion portion)
1a distal end section, bending section
1b endoscope insertion path
1c wire insertion path (deformation-assisting-member insertion path)
1d treatment-tool insertion path (deformation-assisting-member insertion path)
2 guide wire (deformation assisting member)
2b bending section (curved section)
3 gripped part
4 position adjustment part
5 treatment tool (deformation assisting member)
7 outer sheath (deformation assisting member)
10 endoscope
100 medical device

1. A medical device comprising:
a sheath that has a longitudinal axis and that is provided with a flexible section provided at least at a distal end section of the sheath, the flexible section being configured to have flexibility so as to be deformed between a first shape extending along the longitudinal axis and a second shape bent in a direction intersecting the longitudinal axis;
a deformation assisting member having a longitudinal axis extending parallel to the longitudinal axis of the sheath, the deformation assisting member being configured to be attached to the sheath so as to be movable along a surface of the sheath;
a position adjustment part connected to a proximal end section of the sheath, the position adjustment part being configured to adjust a relative position of the deformation assisting member with respect to the sheath; and
a curved section provided at one of the flexible section and one longitudinal section of the deformation assisting member and having a bent shape in a natural state,
wherein the position adjustment part adjusts the relative position of the deformation assisting member with respect to the sheath, thereby causing the deformation assisting member to impart to the flexible section a driving force for causing deformation between the first shape and the second shape, according to an adjustment amount adjusted by the position adjustment part.

2. A medical device according to claim 1, wherein the deformation assisting member has larger rigidity than the rigidity of the flexible section.

3. A medical device according to claim 1,
wherein the flexible section has the first shape in the natural state; and
the curved section is provided at the one longitudinal section of the deformation assisting member.

4. A medical device according to claim 1,
wherein the flexible section has the second shape in the natural state;
the curved section is provided at one section or the entire section of the flexible section; and
the deformation assisting member has a straight shape extending along the longitudinal axis of the deformation assisting member over the entire length.

5. A medical device according to claim 3,
wherein the sheath has a deformation-assisting-member insertion path that penetrates therethrough in a longitudinal direction and into which the deformation assisting member is inserted; and
the deformation assisting member has the curved section at a position away from the distal end section toward the proximal end.

6. A medical device according to claim 5, wherein the deformation assisting member is a treatment tool that has, at a position closer to the distal end than the curved section is, a distal-end operating part configured to treat a living tissue.

7. A medical device according to claim 5, wherein the deformation assisting member is a wire having, at a position closer to the distal end than the curved section is, a loop section that is disposed in a direction intersecting the longitudinal direction of the sheath and that is formed into a loop shape.

8. A medical device according to claim 1,
wherein the sheath has: a deformation-assisting-member insertion path that penetrates therethrough in a longitudinal direction and into which the deformation assisting member is inserted; and an endoscope insertion path that penetrates therethrough in the longitudinal direction and into which an endoscope is inserted; and
the curved section is provided so as to be bent in a plane that includes a central axis of the deformation-assisting-member insertion path and a central axis of the endoscope insertion path.

9. A medical device according to claim 1,
wherein the sheath has an endoscope insertion path that penetrates therethrough in a longitudinal direction and into which an endoscope is inserted; and
a fixing portion configured to fix the endoscope in the endoscope insertion path about a longitudinal axis of the endoscope with respect to the sheath is further provided.
